(19) United States
(12) Patent Application Publication
(10) Pub. No.: US 2024/0308975 A1
(43) Pub. Date: Sep. 19, 2024

(54) POLYMORPHS OF AN SSAO INHIBITOR

(71) Applicant: Eli Lilly and Company, Indianapolis, IN (US)

(72) Inventor: David Michael REMICK, Indianapolis, IN (US)

(21) Appl. No.: 18/675,439

(22) Filed: May 28, 2024

Related U.S. Application Data
(63) Continuation of application No. 18/484,810, filed on Oct. 11, 2023, now abandoned.
Application No. 18/484,810 is a continuation of application No. 17/445,792, filed on Aug. 24, 2021, now Pat. No. 11,820,754.
(60) Provisional application No. 63/070,147, filed on Aug. 25, 2020.

(51) Int. Cl. C07D 401/04; C07C 309/30

(52) U.S. Cl. CPC C07D 401/04; C07C 309/30; C07B 2200/13

(57) ABSTRACT

Provided herein are polymorphs of (2E)-3-fluoro-2-({[2-(4-methoxypiperidin-1-yl)pyrimidin-5 -yl]oxy} methyl)prop-2-en-1-aminium 4-methylbenzenesulfonate, compositions thereof, methods of preparation thereof, and methods of use thereof.

### CROSS-REFERENCE TO RELATED APPLICATIONS

This application is a continuation of U.S. patent Application Ser. No .: 18/484,810, filed Oct. 11, 2023, which is a continuation of U.S. patent application Ser. No.: 17/445,792, filed Aug. 24, 2021, now U.S. Pat. No.: 11,820,754, which claims priority to U.S. Provisional Patent Application No.: 63/070,147, filed Aug. 25, 2020, the disclosures of which are hereby incorporated by reference in their entireties for all purposes.

### FIELD

Provided herein are polymorphs of (2E)-3-fluoro-2-({[2-(4-methoxypiperidin-1yl)pyrimidin-5-yl]oxy} methyl)prop-2-en-1-aminium 4-methylbenzenesulfonate, compositions thereof, methods of preparation thereof, and methods of use thereof.

### BACKGROUND

Semicarbazide-sensitive amino oxidase/vascular adhesion protein-1 (SSAO/VAP-1) is a member of the semicarbazide-sensitive amino oxidase family. SSAO/VAP-1 has been alternatively referred to as VAP-1 or SSAO. SSAO/VAP-1 is an enzyme that exists both as a membrane-bound and a soluble isoform; it is predominantly expressed from endothelial cell surface, vascular smooth muscle and adipose cells. SSAO/VAP-1 participates in many cellular processes including glucose disposition, inflammation responses, and leukocyte recruitment. High activity levels of this enzyme are associated with diabetes, atherosclerosis, strokes, chronic kidney disease, and Alzheimer's disease, among other disorders. Recently SSAO/VAP-1 has been implicated in the pathogenesis of liver disorders such as fatty liver disease. U.S. Pat. No. 10,287,270, the content of which is incorporated herein by reference in its entirety, discloses (2E)-3-fluoro-2-({[2-(4-methoxypiperidin-1-yl)pyrimidin-5-yl]oxy}methyl)prop-2-en-1-amine (designated herein as “Compound I”) and a polymorphic form of (2E)-3-fluoro-2-({[2-(4-methoxypiperidin-1-yl)pyrimidin-5-yl]oxy}methyl)prop-2-en-1-aminium 4-methylbenzenesulfonate ((2E)-3-fluoro-2-({[2-(4-methoxypiperidin-1-yl)pyrimidin-5-yl]oxy}methyl)prop-2-en-1-aminium 4-methylbenzenesulfonate is designated herein as “Compound I tosylate” and the polymorphic form is designated herein as “Form I”), the structures of which are provided below.

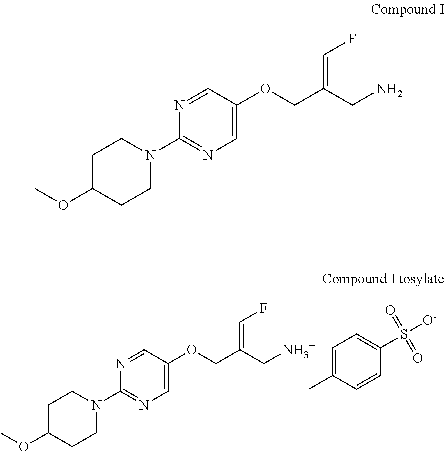 [SMILES: COC1CCN(c2ncc(OC/C(=C/F)CN)cn2)CC1 ; COC1CCN(c2ncc(OC/C(=C/F)C[NH3+])cn2)CC1 ; Cc1ccc(S(=O)(=O)[O-])cc1]

Compound I is a potent SSAO inhibitor being developed as a therapeutic for liver disorders. To move a drug candidate such as Compound I to a viable pharmaceutical product, it can be important to understand whether the drug candidate or its salt form has polymorphic forms, as well as the relative stability and interconversions of these forms under conditions likely to be encountered upon large-scale production, transportation, storage, and pre-usage preparation. The ability to control and produce a stable polymorph with a robust manufacturing process can be key for regulatory approval and marketing. Large-scale production processes for high purity Compound I can be improved by use of particular polymorphic forms. Accordingly, there is a need for various new polymorphic forms of Compound I or its salt form with different chemical and physical stabilities, and compositions and uses of the same.

### BRIEF SUMMARY

In one aspect, provided herein is a polymorph of Compound I tosylate.

In another aspect, provided herein are methods of preparing a polymorph of Compound I tosylate.

In another aspect, provided herein are compositions comprising a polymorph of Compound I tosylate.

In another aspect, provided herein are methods of treating a subject in need of treatment of liver disorders using a polymorph of Compound I tosylate. Also provided is use of a polymorph of Compound I tosylate in the manufacture of a medicament for treating liver disorders.

### DESCRIPTION OF THE DRAWINGS

FIG. 1 shows an X-ray powder diffraction (XRPD) pattern of polymorphic Form II of Compound I tosylate (Form II).

FIG. 2 shows a differential scanning calorimetry (DSC) graph and a thermogravimetric analysis (TGA) graph of Form II.

FIG. 3 shows the results of competitive slurry experiments performed at 25° C. and 50° C.

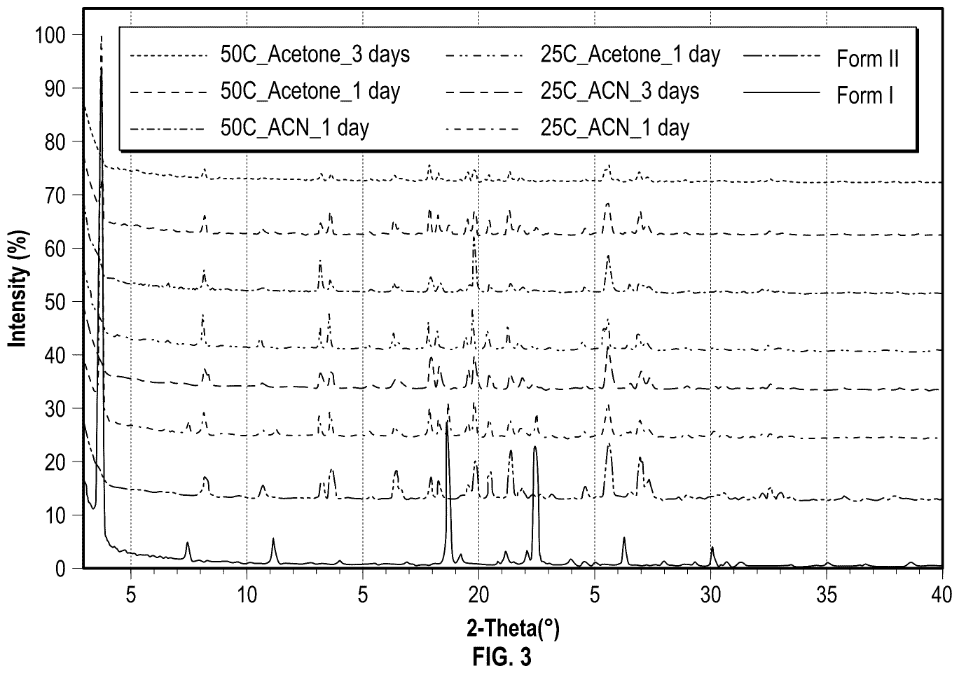

### DETAILED DESCRIPTION

### Definitions

As used herein and in the appended claims, the singular forms “a”, “an” and “the” include plural forms, unless the context clearly dictates otherwise.

As used herein, and unless otherwise specified, the terms “about” and “approximately,” when used in connection with doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in connection with a value, contemplate a variation within #15%, within ±10%, within ±5%, within ±4%, within ±3%, within ±2%, within ±1%, or within ±0.5% of the specified value. Reference to “about” a value or parameter herein includes (and describes) embodiments that are directed to that value or parameter per se. For example, description referring to “about X” includes description of “X”.

As used herein, the term “polymorph” or “polymorphic form” refers to a crystalline form of a compound. Different polymorphs may have different physical properties such as, for example, melting temperatures, heats of fusion, solubilities, dissolution rates, and/or vibrational spectra as a result of the arrangement or conformation of the molecules or ions in the crystal lattice. The differences in physical properties exhibited by polymorphs may affect pharmaceutical parameters, such as storage stability, compressibility, density (important in formulation and product manufacturing), and dissolution rate (an important factor in bioavailability).

As used herein, the term “pharmaceutically acceptable carrier,” and cognates thereof, refers to adjuvants, binders, diluents, etc. known to the skilled artisan that are suitable for administration to an individual (e.g., a mammal or non-mammal). Combinations of two or more carriers are also contemplated. The pharmaceutically acceptable carrier(s) and any additional components, as described herein, should be compatible for use in the intended route of administration (e.g., oral or parenteral) for a particular dosage form, as would be recognized by the skilled artisan.

As used herein, “treatment” or “treating” is an approach for obtaining beneficial or desired results including clinical results. For purposes of this disclosure, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of this disclosure contemplate any one or more of these aspects of treatment.

The term “subject” refers to an animal, including, but are not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

As used herein, the term “therapeutically effective amount” refers to an amount of a compound or composition sufficient to treat a specified disorder, condition or disease such as to ameliorate, to palliate, to lessen, and/or to delay one or more of its symptoms.

As used herein, the term “substantially as shown in” when referring, for example, to an XRPD pattern, a DSC graph, or a TGA graph, includes a pattern or graph that is not necessarily identical to those depicted herein, but falls within the limits of experimental errors or deviations when considered by one of ordinary skill in the art.

As used herein, the term “substantially free of” means that the composition contains the indicated substance or substances in an amount of less than about 25%, less than about 20%. less than about 15%, less than about 10%, less than about 5%, less than about 4%, less than about 3%, less than about 2%, or less than about 1% by weight.

### Polymorphs

In one aspect, provided herein is a polymorph of Compound I tosylate, which has the structure shown below.

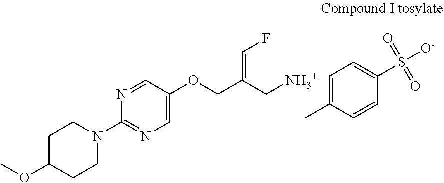 [SMILES: COC1CCN(c2ncc(OC/C(=C/F)C[NH3+])cn2)CC1 ; Cc1ccc(S(=O)(=O)[O-])cc1]

A polymorph of Compound I or a salt thereof may provide the advantages of bioavailability and stability and may be suitable for use as an active agent in a pharmaceutical composition. Variations in the crystal structure of a pharmaceutical drug substance may affect the dissolution rate (which may affect bioavailability, etc.), manufacturability (e.g., ease of handling, ease of purification, ability to consistently prepare doses of known strength, etc.) and stability (e.g., thermal stability, shelf life (including resistance to degradation), etc.) of a pharmaceutical drug product. Such variations may affect the methods of preparation or formulation of pharmaceutical compositions in different dosage or delivery forms, such as solid oral dosage forms including tablets and capsules. Compared to other forms such as non-crystalline or amorphous forms, polymorphs may provide desired or suitable hygroscopicity, particle size control, dissolution rate, solubility, purity, physical and chemical stability, manufacturability, yield, reproducibility, and/or process control. Thus, polymorphs of Compound I may provide advantages of improving the manufacturing process of an active agent or the stability or storability of a drug product form of the active agent, or having suitable bioavailability and/or stability as an active agent.

The use of certain conditions, such as the use of different solvents and/or temperatures, has been found to produce different polymorphs of Compound I or a salt thereof, including polymorphic Form II described herein, which may exhibit one or more favorable characteristics described herein.

### Form II

In some embodiments, provided herein is polymorphic Form II of Compound I tosylate.

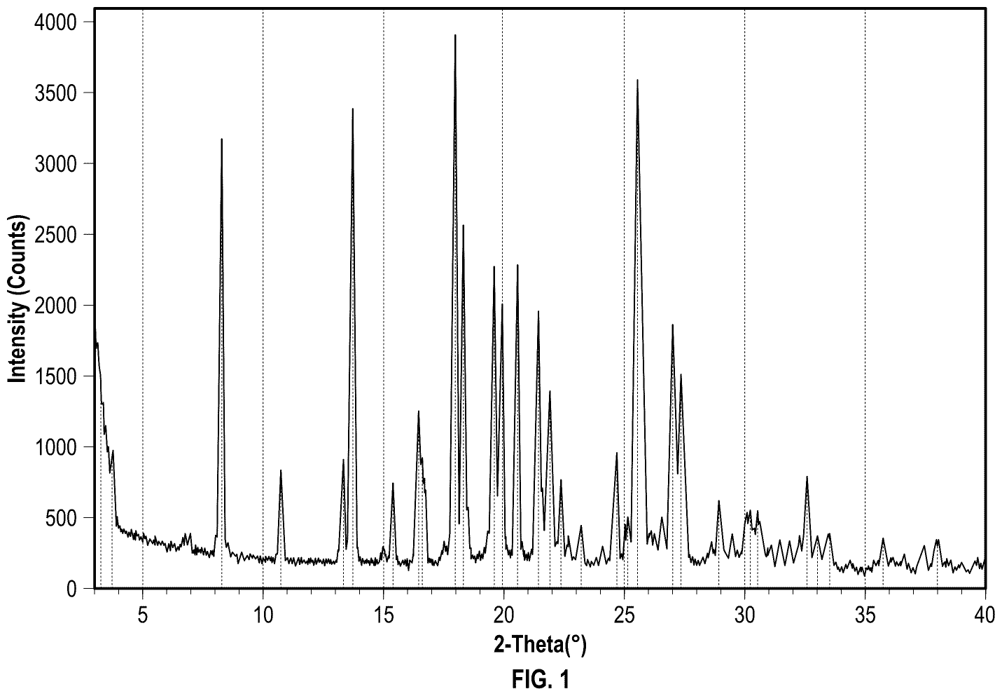

In some embodiments, Form II has an XRPD pattern substantially as shown in FIG. 1. Angles 2-theta and relative peak intensities that may be observed for Form II using XRPD are shown in Table 1.

TABLE 1

| Angle/2θ | Intensity/counts | Intensity % |
|---|---|---|
| 3.2 | 1161 | 31.3 |
| 3.7 | 641 | 17.3 |
| 8.3 | 2944 | 79.4 |
| 10.8 | 635 | 17.1 |
| 13.3 | 729 | 19.7 |
| 13.7 | 3216 | 86.8 |
| 15.4 | 554 | 14.9 |
| 16.5 | 1065 | 28.7 |
| 16.6 | 623 | 16.8 |
| 18.0 | 3707 | 100 |
| 18.3 | 2375 | 64.1 |
| 19.6 | 2048 | 55.2 |
| 19.9 | 1792 | 48.3 |
| 20.6 | 2058 | 55.5 |
| 21.4 | 1735 | 46.8 |
| 21.9 | 1170 | 31.6 |
| 22.4 | 575 | 15.5 |
| 23.2 | 236 | 6.4 |
| 24.7 | 702 | 18.9 |
| 25.1 | 327 | 8.8 |
| 25.5 | 3281 | 88.5 |
| 27.0 | 1680 | 45.3 |
| 27.3 | 1331 | 35.9 |
| 28.9 | 397 | 10.7 |
| 30.2 | 323 | 8.7 |
| 30.5 | 312 | 8.4 |
| 32.6 | 605 | 16.3 |
| 33.5 | 212 | 5.7 |
| 35.7 | 221 | 6 |
| 38.0 | 203 | 5.5 |

In some embodiments, Form II has an XRPD pattern displaying at least two, at least three, at least four, at least five, at least six, at least seven, at least eight, at least nine, at least ten, at least fifteen, or at least twenty of the peaks at angles 2-theta with the greatest intensity in the XRPD pattern as shown in FIG. 1 or as provided in Table 1. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein can vary by ±0.6 degrees, ±0.4 degrees, ±0.2 degrees, or ±0.1 degrees 2-theta. In some embodiments, peak assignments listed herein, including for Form II, can vary by ±0.6 degrees 2-theta. In some embodiments, peak assignments listed herein can vary by ±0.4 degrees 2-theta. In some embodiments, peak assignments listed herein can vary by ±0.2 degrees 2-theta. In some embodiments, peak assignments listed herein can vary by ±0.1 degrees 2-theta.

In some embodiments, Form II has an XRPD pattern comprising peaks at angles 2-theta of 13.7±0.2. 18.0'0.2, and 25.5±0.2 degrees. In some embodiments, Form II has an XRPD pattern comprising peaks at angles 2-theta of 8.3±0.2, 13.7±0.2, 18.0±0.2, 18.3±0.2, and 25.5±0.2 degrees. In some embodiments, Form II has an XRPD pattern comprising peaks at angles 2-theta of 8.3±0.2, 13.7±0.2, 18.0±0.2, 18.3±0.2, 19.6±0.2, 20.6±0.2, and 25.5±0.2 degrees. In some embodiments, Form II has an XRPD pattern comprising peaks at angles 2-theta of 8.3±0.2. 13.7±0.2, 18.0±0.2, 18.3±0.2, 19.6±0.2, 19.9±0.2, 20.6±0.2, 21.4±0.2, 25.5±0.2, and 27.0±0.2 degrees.

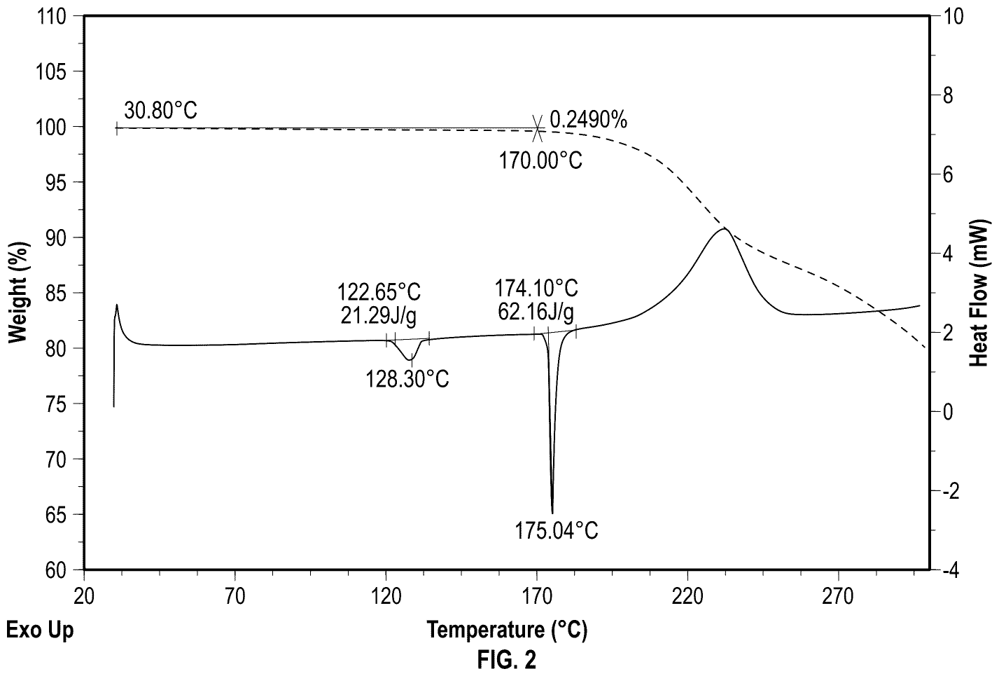

In some embodiments, Form II has a DSC graph substantially as shown in FIG. 2. In some embodiments, Form II is characterized as having an endotherm onset at about 123° C. and/or an endotherm onset at about 174° C. as determined by DSC. In some embodiments, Form II is characterized as having an endotherm onset at 123±5° C., 123±4° C., 123±3° C., 123±2° C., or 123±1° C. as determined by DSC. In some embodiments, Form II is characterized as having an endotherm onset at 174±5° C., 174±4° C., 174±3° C., 174±2° C., or 174±1° C.

In some embodiments, Form II has a TGA graph substantially as shown in FIG. 2.

In some embodiments of Form II, at least one, at least two, at least three, at least four, or all of the following (a)-(e) apply:
(a) Form II has an XRPD pattern comprising peaks at angles 2-theta of 13.7±0.2, 18.0±0.2, and 25.5±0.2 degrees; an XRPD pattern comprising peaks at angles 2-theta of 8.3±0.2, 13.7±0.2, 18.0±0.2, 18.3±0.2, and 25.5±0.2 degrees; an XRPD pattern comprising peaks at angles 2-theta of 8.3±0.2, 13.7±0.2, 18.0±0.2, 18.3±0.2, 19.6±0.2, 20.6±0.2, and 25.5±0.2 degrees; or an XRPD pattern comprising peaks at angles 2-theta of 8.3±0.2, 13.7±0.2, 18.0±0.2, 18.3±0.2, 19.6±0.2, 19.9±0.2, 20.6±0.2, 21.4±0.2, 25.5±0.2, and 27.0±0.2 degrees;
(b) Form II has an XRPD pattern substantially as shown in FIG. 1;
(c) Form II is characterized as having an endotherm onset at about 123° C. and/or an endotherm onset at about 174° C. as determined by DSC;
(d) Form II has a DSC graph substantially as shown in FIG. 2; and
(e) Form II has a TGA graph substantially as shown in FIG. 2.

### Compositions

In another aspect, provided herein is a composition comprising a polymorphic form disclosed herein. In some embodiments, the composition comprises Form II. In some embodiments, the composition is a pharmaceutical composition. In some embodiments, the composition further comprises a pharmaceutically acceptable carrier. In some embodiments, the composition comprising Form II is substantially free of Form I. In some embodiments, the composition is substantially free of amorphous or non-crystalline form of Compound I.

In some embodiments of the composition comprising Form II, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Form II. In some embodiments of the composition comprising Form II, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound I exists in Form II.

In some embodiments, provided is a tablet or capsule comprising substantially pure Form II, and a pharmaceutically acceptable carrier.

### Methods of Preparation

### Form II

In some embodiments, provided is a method of preparing Form II, comprising evaporating a solution of Compound I tosylate in a solvent, wherein the solvent comprises acetonitrile (ACN) and water. In some embodiments, the volume ratio of ACN to water is between about 95:5 and about 5:95. In some embodiments, the volume ratio of ACN to water is about 95:5, about 90:10, about 80:20, about 70:30, about 60:40, about 50:50, about 40:60, about 30:70, about 20:80, about 10:90, or about 5:95. In some embodiments, the volume ratio of ACN to water is about 95:5.

In some embodiments, provided is a method of preparing Form II, comprising stirring a mixture comprising Compound I tosylate and a solvent, wherein the solvent comprises ACN and water or the solvent comprises ethyl acetate. In some embodiments, the solvent comprises ethyl acetate. In some embodiments, the solvent comprises ACN and water. In some embodiments, the volume ratio of ACN to water is between about 95:5 and about 5:95. In some embodiments, the volume ratio of ACN to water is about 95:5, about 90:10, about 80:20, about 70:30, about 60:40, about 50:50, about 40:60, about 30:70, about 20:80, about 10:90, or about 5:95. In some embodiments, the volume ratio of ACN to water is about 95:5.

In some embodiments, provided is a method of preparing Form II, comprising adding an anti-solvent to a solution of Compound I tosylate in a solvent, wherein the solvent comprises ACN and water and wherein the anti-solvent comprises 2-methyltetrahydrofuran (2-MeTHF).

### Methods of Use

In another aspect, provided herein is a method of treating a liver disorder in a patient (e.g., a human patient) in need thereof comprising administering a therapeutically effective amount of Form II. In some embodiments, the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the liver disorder is NAFLD or NASH. In some embodiments, the liver disorder is NAFLD. In some embodiments, the liver disorder is NASH. In some embodiments, the patient has had a liver biopsy. In some embodiments, the method further comprises obtaining the results of a liver biopsy.

In some embodiments, provided is a method of impeding or slowing the progression of NAFLD to NASH in a patient (e.g., a human patient) in need thereof comprising administering a therapeutically effective amount of Form II.

### Methods of Manufacturing a Medicament

In some embodiments, provided is use of Form II in the manufacture of a medicament for use in a method disclosed herein.

### Kits

Also provided are articles of manufacture and kits comprising any of the polymorphic forms or compositions provided herein. The article of manufacture may comprise a container with a label. Suitable containers include, but are not limited to, bottles, vials, and test tubes. The containers may be formed from a variety of materials such as glass or plastic. The container may hold a pharmaceutical composition provided herein. The label on the container may indicate that the pharmaceutical composition is used for treating a condition described herein, and may also indicate directions for either in vivo or in vitro use.

In one aspect, provided herein are kits comprising a polymorphic form or composition described herein and instructions for use. A kit may additionally contain any materials or equipment that may be used in the administration of the polymorphic forms or composition, such as vials, syringes, or IV bags. A kit may also contain sterile packaging.

### EXAMPLES

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

The following abbreviations may be used herein:

| | |
|---|---|
| XRPD | X-Ray Powder Diffraction |
| DSC | Differential Scanning Calorimetry |
| TGA | Thermogravimetric Analysis |
| RT | Room Temperature |
| ACN | Acetonitrile |
| EA | Ethyl Acetate |
| 2-MeTHF | 2-Methyltetrahydrofuran |
| MeOH | Methanol |
| THF | Tetrahydrofuran |
| TsOH | p-Toluenesulfonic Acid |
| TsOH•H2O | p-Toluenesulfonic Acid Monohydrate |
| MIBK | Methyl Isobutyl Ketone |
| MTBE | Methyl Tert-butyl Ether |

The polymorphic forms of Compound I tosylate were characterized by various analytical techniques, including XRPD, DSC, and TGA, using the procedures described below.

### XRPD

XRPD analyses were performed with techniques and equipments known in the art with Cu K-alpha radiation.

### DSC

DSC analyses were performed with techniques and equipments known in the art. Each sample was heated from 30° C. to 300° C. at a rate of 10° C./min.

### TGA

TGA experiments were performed with techniques and equipments known in the art. Each sample was heated from RT to 300° C. at a rate of 10° C./min.

### Example 1. Preparation of Form II

Solution of Compound I tosylate in different solvents were prepared at room temperature and filtered through 0.45 μm nylon syringe filter into clean vessels. Solvents were evaporated in the fume hood for 3 days. All solids were characterized by XRPD. The results are provided in Table 2.

TABLE 2

| Solvent | Result |
|---|---|
| MeOH | Form I |
| Acetone | Form I |
| ACN:water (95:5, v/v) | Form II |
| THF:water (95:5, v/v) | Form I |

Solution of Compound I tosylate in different solvents were prepared at room temperature (50° C. for acetone) and filtered through 0.45 μm nylon syringe filter into clean vessels. Different anti-solvents were added to precipitate out solids. The mixtures were placed in the fume hood for 3 days. The samples were centrifuged and the supernatants were removed. All solids were characterized by XRPD. The results are provided in Table 3.

TABLE 3

| | Target Con. | | |
|---|---|---|---|
| Solvent | (mg/mL) | Anti-solvent | Result |
| MeOH | 200 | MIBK | Form I |
| | | EA | Form I |
| | | 2-MeTHF | Form I |
| | | MTBE | Form I |
| | | Heptane | — |
| Acetone | 25 | Toluene | Form I |
| | | EA | Form I |
| | | 2-MeTHF | Form I |
| | | MTBE | Form I |
| | | Heptane | Form I * |
| ACN:water (95:5, v/v) | 100 | MIBK | Form I |
| | | EA | Form I |
| | | 2-MeTHF | Form II |
| | | MTBE | Form I |
| | | Heptane | — |
| THF:water (95:5) | 100 | MIBK | Form I |
| | | EA | Form I |
| | | Toluene | Form I |
| | | MTBE | Form I |
| | | Heptane | — |

*: minor difference due to the crystal habit.
—: Clear solution or oil

Form II was analyzed by XRPD, DSC, and TGA. FIG. 1 shows an XRPD pattern of Form II. FIG. 2 shows a DSC graph of Form II. As shown in the DSC graph, an endotherm onset at about 123° C. and an endotherm onset at about 174° C. were observed. FIG. 2 also shows a TGA graph of Form II.

### Example 2. Large-scale Preparation of Form II

Compound I in EA solution (20.70 kg, 10.7% w/w) was charged in a 1000 L glass lined reactor. TsOH in EA solution (prepared by charging 13.60 kg of TsOH·H₂O and 75 kg of EA in a 500 L glass lined reactor and stirred at 35-40° C. for 30-60 minutes) was charged slowly at 20-25° C. in 2-4 hours. The mixture was stirred at 20-25° C. for 5-8 hours and then filtered via a 250 L Hastelloy alloy dryer. The wet cake was rinsed with EA and dried. The resulting solid was characterized by XRPD and confirmed to be Form II.

### Example 3. Competitive Slurry Study

Competitive slurry experiments were performed at 25° C. and 50° C. Saturated solutions of Form I in ACN and acetone at 25° C. and 50°° C. were prepared. About 15 mg of Form I and Form II were weighted into the solutions respectively, and the suspensions were slurried for 1 or 3 days at 800 rpm. As determined by XRPD, a mixture of Form I and Form II could be completely converted to Form II at both temperatures, indicating that Form II was more thermodynamically stable at a temperature of 50° C. or below. The results are summarized in Table 4.

TABLE 4

| Starting | | Temperature | Slurry | XRPD Pattern |
|---|---|---|---|---|
| Material | Solvent | (° C.) | time (days) | (wet cake) |
| Form I + | ACN | 25 | 1 | Form I + Form II |
| Form II | | | 3 | Form II |
| | | 50 | 1 | Form II |
| | Acetone | 25 | 1 | Form II |
| | | 50 | 1 | Form I + Form II |
| | | | 3 | Form II |

All documents, including patents, patent application and publications cited herein, including all documents cited therein, tables, and drawings, are hereby expressly incorporated by reference in their entirety for all purposes.

While the foregoing written description of the polymorphic forms, uses, and methods described herein enables one of ordinary skill in the art to make and use the polymorphic forms, uses, and methods described herein, those of ordinary skill in the art will understand and appreciate the existence of variations, combinations, and equivalents of the specific embodiments, methods, and examples herein.

1. A polymorph of (2E)-3-fluoro-2-({[2-(4-methoxypiperidin-1-yl)pyrimidin-5-yl]oxy}methyl)prop-2-en-1-aminium 4-methylbenzenesulfonate: 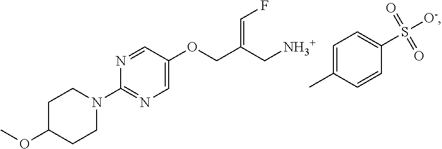
characterized as having an X-ray powder diffraction (XRPD) pattern comprising peaks at angles 2-theta of 13.7±0.2, 18.0±0.2, and 25.5±0.2 degrees.

2. The polymorph of claim 1, characterized as having an XRPD pattern comprising peaks at angles 2-theta of 8.3±0.2, 13.7±0.2, 18.0±0.2, 18.3±0.2, and 25.5±0.2 degrees.

3. The polymorph of claim 1 or 2, characterized as having an XRPD pattern substantially as shown in FIG. 1.

4. The polymorph of any one of claims 1-3, characterized as having an endotherm onset at about 123° C. and/or an endotherm onset at about 174° C. as determined by differential scanning calorimetry (DSC).

5. The polymorph of any one of claims 1-4, characterized as having a DSC graph substantially a shown in FIG. 2.

6. A method of preparing the polymorph of any one of claims 1-5, comprising:
evaporating a solution of (2E)-3-fluoro-2-({[2-(4-methoxypiperidin-1-yl)pyrimidin-5-yl]oxy}methyl)prop-2-en-1-aminium 4-methylbenzenesulfonate in a solvent, wherein the solvent comprises acetonitrile (ACN) and water;
stirring a mixture comprising (2E)-3-fluoro-2-({[2-(4-methoxypiperidin-1-yl)pyrimidin-5-yl]oxy}methyl)prop-2-en-1-aminium 4-methylbenzenesulfonate and a solvent, wherein the solvent comprises ACN and water or the solvent comprises ethyl acetate; or adding an anti-solvent to a solution of (2E)-3-fluoro-2-({[2-(4-methoxypiperidin-1-yl)pyrimidin-5-yl]oxy}methyl)prop-2-en-1-aminium 4-methylbenzenesulfonate in a solvent,
wherein the solvent comprises ACN and water and wherein the anti-solvent comprises 2-methyltetrahydrofuran (2-MeTHF).

7. The method of claim 6, wherein the volume ratio of ACN to water is about 95:5.

8. A pharmaceutical composition comprising the polymorph of any one of claims 1-5, and a pharmaceutically acceptable carrier.

9. The pharmaceutical composition of claim 8, wherein the pharmaceutical composition is substantially free of other polymorphic forms of (2E)-3-fluoro-2-({[2-(4-methoxypiperidin-1-yl)pyrimidin-5-yl]oxy}methyl)prop-2-en-1-amine or a salt thereof.

10. A method of treating a liver disorder in a subject in need thereof, comprising administering a therapeutically effective amount of the polymorph of any one of claims 1-5 or the pharmaceutical composition of claim 8 or 9.

11. The method of claim 10, wherein the liver disorder is liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), or non-alcoholic steatohepatitis (NASH).

12. Use of the polymorph of any one of claims 1-5 in the manufacture of a medicament for treating a liver disorder.

13. The use of claim 12, wherein the liver disorder is liver inflammation, liver fibrosis, alcohol induced fibrosis, steatosis, alcoholic steatosis, PSC, PBC, NAFLD, or NASH.
